(19) United States
(12) Patent Application Publication
(10) Pub. No.: US 2007/0213641 A1
(43) Pub. Date: Sep. 13, 2007

(54) Constrained balloon disc sizer

(75) Inventor: Tom J. Francis, Cordova, TN (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: SDGI HOLDINGS, INC.

(21) Appl. No.: 11/349,279

(22) Filed: Feb. 8, 2006

(51) Int. Cl. A61B 5/103

(52) U.S. Cl. 600/594

(57) ABSTRACT

An intradisc sizer is provided. The intradisc sizer includes a constrained expandable member, a longitudinal element, and a dispensing device. A fluid may be used to inflate the constrained expandable member, which is placed in an intradiscal space. Methods of using the intradisc sizer and implanting a spinal implant, and a kit containing the intradisc sizer, also are provided.

### FIELD OF THE INVENTION

Embodiments relate to methods and systems for characterizing the intradiscal space. More particularly, embodiments of the invention relate to methods and systems for determining the volume, geometry, and other parameters of the intradiscal space using constrained expandable members inflated with a fluid.

### BACKGROUND OF THE INVENTION

The intervertebral disc functions to stabilize the spine and to distribute forces between vertebral bodies. The intervertebral disc primarily includes three structures: the nucleus pulposus, the annulus fibrosis, and two vertebral end-plates. The nucleus pulposus is an amorphous hydrogel in the center of the intervertebral disc. The annulus fibrosis, which is comprised mostly of highly structured collagen fibers, maintains the nucleus pulposus within the center of the intervertebral disc. The vertebral end-plates, primarily comprised of hyalin cartilage, separate the disc from adjacent vertebral bodies and act as a transition zone between the hard vertebral bodies and the soft disc.

Intervertebral discs may be displaced or damaged due to trauma, disease, and the normal aging process. One way to treat a displaced or damaged intervertebral disc is by surgical removal of a portion or all of the intervertebral disc, including the nucleus and the annulus fibrosis. However, the removal of the damaged or unhealthy disc may allow the disc space to collapse, which may lead to instability of the spine, abnormal joint mechanics, nerve damage, and severe pain. Therefore, after removal of the disc, a spinal implant such as a prosthetic nucleus, artificial disc, or fusion cage may be implanted in order to replace the removed nucleus or annulus, or a portion thereof.

Because the spinal implant is replacing all or part of the intervertebral disc, it may be desirable to select the spinal implant according to the natural dimensions and geometry of the intervertebral disc that is to be replaced or augmented.

The description herein of problems and disadvantages of known devices and methods is not intended to limit the invention to the exclusion of these known entities. Indeed, embodiments of the invention may include one or more of the known devices and methods without suffering from the disadvantages and problems noted herein.

### SUMMARY OF THE INVENTION

What is needed are systems and methods for determining various parameters of the intervertebral disc space, such as the volume, dimensions, and geometry. Embodiments of the invention solve some or all of these needs, as well as additional needs.

Therefore, in accordance with one embodiment, an intradisc sizer is provided for determining at least one parameter of an intradiscal space. The intradisc sizer comprises a longitudinal element, a constrained expandable member, and a dispensing device. The longitudinal element has distal and proximate ends and an axially concentric bore. The constrained expandable member comprises an internal cavity and is connected to and in fluid communication with the distal end of the longitudinal element. The dispensing device is capable of holding a fluid and is adapted to be connected to and in fluid communication with the proximate end of the longitudinal element.

In another embodiment, a kit is provided for determining at least one parameter of an intradiscal space. The kit comprises a longitudinal element having distal and proximate ends and an axially concentric bore. The kit further comprises a constrained expandable member having an internal cavity. The constrained expandable member is capable of being connected to, and in fluid communication with, the distal end of the longitudinal element. Also, the kit may comprise a dispensing device capable of holding a fluid. The dispensing device is capable of being connected to, and in fluid communication with, the proximate end of the longitudinal element.

In a further embodiment, there is provided a method for determining at least one parameter of an intradiscal space. The method comprises inserting a constrained expandable member into the intradiscal space. The constrained expandable member of the intradisc sizer can be inflated with fluid. The volume of fluid used to inflate the constrained expandable member can be measured. Finally, the constrained expandable member is deflated and removed from the intradiscal space.

In still another embodiment, there is provided a method of implanting a spinal implant in an intradiscal space. At least a portion of a nucleus of an intervertebral disc is removed. A constrained expandable member is inserted into the intradiscal space and inflated with a fluid. The volume of fluid used to inflate the constrained expandable member can be measured. The constrained expandable member is deflated and removed from the intradiscal space. Finally, a spinal implant may be selected based on the volume of fluid used to inflate the expandable member, and then implanted.

These and other features and advantages of the embodiments will be apparent from the description provide herein.

### BRIEF DESCRIPTION OF THE DRAWINGS

FIG. 1 is an illustration of a preferred device according to one embodiment.

FIG. 2 is an illustration of the distal portion of the device shown in FIG. 1.

FIG. 3 is an illustration of exemplary planar views of a constrained expandable member according to embodiments of the invention.

FIG. 4 includes an illustration of a preferred method of using the constrained expandable member intradisc sizer.

### DETAILED DESCRIPTION OF THE EMBODIMENTS

The following description is intended to convey a thorough understanding of the various embodiments by providing a number of specifically preferred embodiments and details involving devices and methods for determining one or more parameters of an intradiscal space. It is understood, however, that the invention is not limited to these specific embodiments and details, which are exemplary only. It is further understood that one possessing ordinary skill in the art, in light of known systems and methods, would appreciate the use of the invention for its intended purposes and benefits in any number of alternative embodiments.

Throughout this description, the expression “intradiscal space” may refer to any volume or void between two adjacent vertebrae. The intradiscal space may be the volume inside of the annulus fibrosis of the intervertebral disc. Alternatively, the intradiscal space also may include the annulus fibrosis itself. The intradiscal space also may include only a portion of the volume between two adjacent vertebrae.

The expression “intradisc sizer” refers to a device for determining parameters of an intradiscal space. Parameters of a intradiscal space that may be determined or measured using an intradisc sizer include, but are not limited to, the intradiscal space's volume, general shape, endplate geometry, and so forth. Thus, an intradisc sizer may be useful in characterizing an intradiscal space.

The expression “fluid communication” means that the bodies or elements in fluid communication with each other at least are capable of being in fluid communication. The bodies or elements need not be in fluid communication at all times so long as they are at least capable of being in fluid communication, so that at least when they are in fluid communication, fluid can flow between the respective bodies. The term “fluid” is used herein to denote any flowable material, such as liquids, gases, slurries, suspensions, gels, and the like.

The expression “constrained expandable member” denotes an expandable member that has been constrained so that it does not expand in a uniform manner. For example, a balloon typically is spherical and expands approximately uniformly in all directions as it is inflated, so that its spherical shape is maintained. In a constrained expandable member, however, constraints may be placed anywhere along the outer or inner surface of the expandable member to prevent or retard expansion in one or more directions. For example, the expandable member can be designed in a football shape with constraints on the upper and lower portions of the expandable member (i.e., lateral portions) so that, when inflated, the expandable member expands longitudinally, but experiences little or no expansion in lateral directions. Various methods of making constrained expandable members are known and described in the art.

It is a feature of an embodiment to provide an intradisc sizer for determining at least one parameter of an intradiscal space. The intradisc sizer preferably comprises a longitudinal element, a constrained expandable member, and a dispensing device. The longitudinal element has distal and proximate ends and an axially concentric bore. The constrained expandable member comprises an internal cavity and preferably is connected to, and in fluid communication with, the distal end of the longitudinal element. The dispensing device is capable of holding a fluid and preferably is adapted to be connected to, and in fluid communication with, the proximate end of the longitudinal element.

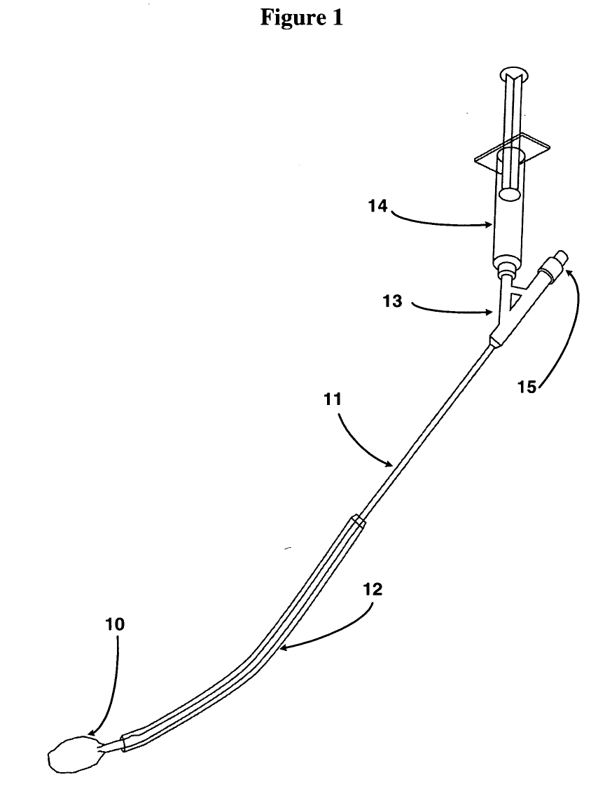

FIG. 1 is an illustration of an exemplary device according to an embodiment.

The exemplary device comprises a constrained expandable member 10 connected to, and in fluid communication with, the distal end of an axially concentric bore in a longitudinal element 11. An optional guide shaft 12 is approximately coaxial to the longitudinal element 11 and sheathes at least a portion of the longitudinal element 11 and constrained expandable member 10. As can be seen in FIG. 1, the distal end of the longitudinal element 11 and constrained expandable member 10 may be extended beyond the distal end of the guide shaft 12. This may be accomplished simply by holding the guide shaft 12 while pushing, inserting, or displacing the longitudinal element 11 and constrained expandable member 10. A syringe 14 or other dispensing device is connected to, and in fluid communication with, the proximate end of the axially concentric bore of the longitudinal element 11 and acts as a dispensing device. An optional Y-adapter 13 may be positioned between the proximate end of the longitudinal element 11 and the syringe 14 to assist in connecting the two components. The Y-adapter 13 also provides an additional, optional access or med-port 15 .

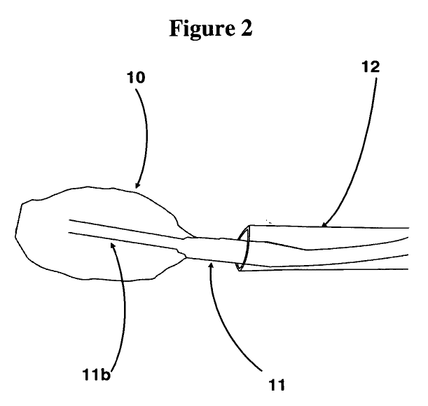

FIG. 2 is an illustration of the distal end of the device shown in FIG. 1. A constrained expandable member 10 is connected to, and in fluid communication with, the distal end of a longitudinal element 11. The distal tip of the longitudinal element lib optionally extends into the constrained expandable member 10. An optional guide shaft 12 is approximately coaxial to the longitudinal element 11. As can be seen, the constrained expandable member 10 and distal end of the longitudinal element 11 are capable of extending beyond the distal end of the optional guide shaft 12. This may be advantageous in order to facilitate delivery of the constrained expandable member 10 to the intradiscal space.

The longitudinal element may be used to push or insert the constrained expandable member into an intradiscal space. Additionally, the longitudinal element may be used to conduct a fluid, such as a saline solution, air, or an imaging contrast medium, from the dispensing device to expand or inflate the constrained expandable member. Because the longitudinal element has an axially concentric bore, it may be described as a shaft or tube such as a cannula, catheter, or trocar. However, the longitudinal element need not be limited to a circular cross section like traditional cannulas, catheters, and trocars. Rectangular, square, elliptical, and other cross-sectional geometries also are contemplated for the longitudinal element. The longitudinal element may be made of any appropriate material, including but not limited to medical plastics such as polyvinyl chlorides, polypropylenes, polystyrenes, acetal copolymers, polyphenyl sulfones, polycarbonates, acrylics, silicone polymers, and mixtures and combinations thereof, and medical alloys. Preferably, the longitudinal element has sufficient biocompatibility to avoid undesirable interactions during its relatively brief insertion into the body.

The longitudinal element may be used to deliver a fluid to the internal cavity of the expandable member. The longitudinal element may have an optimal stiffness and flexibility to facilitate insertion into the body and maneuverability. In a preferred embodiment, the distal end of the longitudinal element may be curved or easily deformable to conform to the intervertebral disc space. Even more preferably, the longitudinal element is capable of being selectively pivoted, or otherwise moving, between a linear and a curved configuration, particularly at its distal end.

Additionally, the longitudinal element may have an optimal diameter for insertion into the body and delivery of the expandable member to the intervertebral disc space. It may be preferable that the diameter of the longitudinal element be not more than the height of the disc space, for example no more than about 12 mm, preferably no more than about 10 mm, and most preferably no more than about 8 mm in diameter. This may allow the longitudinal element to be inserted into the intervertebral disc space for delivery of the expandable member therein. One who is skilled in the art will appreciate how to choose the appropriate size and flexibility of the longitudinal element in accordance with the guidelines described herein.

Constrained expandable members are known, and have commonly been used to either compact cancellous bone or to distract the vertebral bodies. Use of constrained expandable members is disclosed in, for example, U.S. Pat. Nos. 5,972,015; 6,235,043; 6,423,083; 6,607,544; 6,623,505; 6,716,216; 6,719,773; 6,863,672, and U.S. Patent Application Publication Nos. 2001/0011174; 2002/0013600; 2002/0082608; 2002/0099384; 2002/0156482; 2002/0183778; 2003/0032963; 2003/0195547; 2004/0010263; 2004/0225296; and 2004/0167271, the disclosures of each of which are incorporated by reference herein in their entirety.

The constrained expandable member may be connected to and in fluid communication with the distal end of the longitudinal element. The constrained expandable member may be any appropriate biocompatible and inflatable member having an internal cavity. Because the constrained expandable member preferably is inserted into the body only for a momentary period of time, the constrained expandable member need not be as biocompatible as a permanent implant. It may be preferable that the constrained expandable member have sufficient biocompatibility, however, to avoid undesirable interactions during its relatively brief insertion into the body.

The constrained expandable member preferably may be selected to withstand the pressure of inflation when the fluid is delivered to it so as to avoid rupture when inflated. Rupture could cause a leak of the fluid, inaccurate measurement of intradisc characteristics, and consequently should be avoided. Where the fluid is potentially toxic (e.g., an imaging contrast medium), the potential for leakage is of even greater concern, and hence the constrained expandable member may be selected accordingly.

In a preferred embodiment, the constrained expandable member may be made of various polymeric materials such as polyethylene terephthalates, polyolefins, polyurethanes, nylon, polyvinyl chloride, silicone, polyetherketone, polylactide, polyglycolide, poly(lactide-co-glycolide), poly(dioxanone), poly([epsilon]-caprolactone), poly(hydroxylbutyrate), poly(hydroxylvalerate), tyrosine-based polycarbonate, polypropylene fumarate, rubber-based materials and latex, and mixtures and combinations thereof. Because it is contemplated that the constrained expandable member may be inflated with imaging contrast agents and/or radioactive materials, it is preferred to fabricate the expandable member from chemical-resistant materials. In addition, the constrained expandable member may be made from a multi-layered material with an inner chemically-resistant layer, and/or the interior of the constrained member may be coated with a chemically-resistant coating.

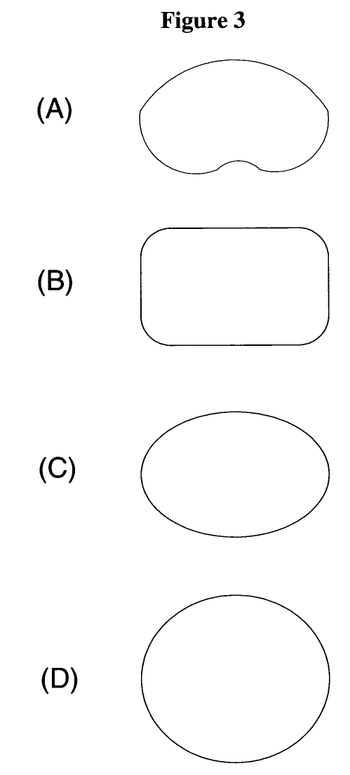

The expansion of the constrained expandable member is limited during inflation or expansion so that the expandable member expands preferentially in certain directions when inflated. For example, a constrained expandable member may have a planar shape such that, once the planar shape has been reached during inflation, continued inflation of the expandable member leads to an increase in height of the expandable member, but does not significantly distort the planar shape of the expandable member. In other words, the profile of the expandable member may be at least partially constrained during inflation whereas the height is variable. FIG. 3, embodiments A, B, C, and D, illustrates exemplary planar shapes of the constrained expandable member. Embodiment A depicts an exemplary kidney-like shape intended to be similar to the shape of the intradiscal space or the shape of an implant. Embodiment B depicts a rectangle with rounded edges. Embodiment C depicts an ellipse. Embodiment D depicts a circle. Constrained expandable members according to the embodiments may have any of these exemplary planar shapes, in addition to other planar shapes that will be appreciated by one of skill in the art, including a circle, ellipse, rectangle with rounded corners, kidney, and “C”-shaped. It is appreciated that at least some level of inflation may be required before the constrained expandable member reaches its constrained planar shape.

In a preferred embodiment, the constrained expandable member may be shaped like an spinal implant, such as a spinal fusion device, a nucleus replacement device, a spinal arthroplasty device, and so forth. A constrained expandable member with a shape similar to a spinal implant may be especially useful for determining if a particular spinal implant is appropriate for use in an intradiscal space and for determining the appropriate size for the spinal implant. For example, a constrained expandable member with a shape similar to that of a spinal implant may be inserted into an intradiscal space and inflated. The suitability of the spinal implant for the intradiscal space in which it is to be implanted may be judged by determining if the constrained expandable member was able to inflate fully in the intradiscal space. An inability of the constrained expandable member to inflate fully may indicate that the correspondingly shaped spinal implant will not fit in the intradiscal space. Constrained expandable members such as those described herein can be fabricated by one of skill in the art.

A dispensing device may be used to draw a fluid such as a saline solution, air, or an imaging contrast medium from a separate container and then deliver the fluid to the longitudinal element, and from the longitudinal element to the constrained expandable member. Preferred dispensing devices include syringes. In a preferred embodiment, the dispensing device may be a syringe graduated by volume so that the volume of fluid in the dispensing device before inflation of the constrained member can easily be measured and compared to the volume of fluid in the dispensing device when the constrained member is experiencing expansion or has been expanded to a maximum volume inside of the intradiscal space. The volume of fluid delivered to the constrained expandable member may be determined by comparing these two values.

The dispensing device may be capable of being detachably connected to, and in fluid communication with, the proximate end of the longitudinal element. The dispensing device may be detachably connected to the longitudinal element using any appropriate detachment means. In a preferred embodiment, the dispensing device may be connected to the proximate end of the longitudinal element using a luer lock. Alternatively, the proximate end of the longitudinal element may include a seal that can be repeatedly punctured by a needle on the dispensing device, if so equipped, much like a medicine-containing vial. Other detachment devices including, but not limited to, luer slip connectors also may be used to detachably connect the dispensing device to the proximate end of the longitudinal element. It is preferred that the detachment device maintain a sufficiently positive connection between the dispensing device and the proximate end of the axially concentric bore of the longitudinal element so as to prevent leakage of the fluid during the rise in pressure of the fluid that may accompany the inflation of the constrained expandable member.

Any applicable fluid may be used to inflate the constrained expandable member, including water, saline solutions, air, and imaging contrast mediums. Imaging contrast mediums may be especially preferred because delivery of the contrast medium to the expandable member may enhance the quality of images taken of the intradiscal space taken during inflation of the member.

Imaging contrast mediums contemplated for use in the embodiments include all applicable imaging contrast mediums, including contrast agents for X-ray, CT, MRI, and PET imaging. Typically, the imaging contrast medium may be chosen to correspond to the imaging technique to be used. For example, if X-ray images are to be taken of the inflated constrained expandable member, then X-ray imaging contrast mediums preferably may be used, and so forth for other imaging procedures (e.g., MRI, CT, and PET scans). Additionally, it may be preferable that the imaging contrast medium comprise a fluid or liquid solution, gel, paste, or suspension of an X-ray, CT, MRI, or PET contrast agent rather than a pure composition of the contrast agent. Therefore, it should be understood that the expression “imaging contrast mediums” includes fluid or liquid solutions, gels, pastes, and suspensions of X-ray, CT, MRI, and PET contrast agents, in addition to pure compositions of the contrast agents. One who is skilled in the art will appreciate the wide variety of imaging contrast mediums that may be used in accordance with the embodiments described herein.

Specific X-ray imaging contrast mediums contemplated for use herein include, but are not limited to, barium sulfate, acetrizoic acid derivatives, diatrizoic acid derivatives such as Hypaque® (commercially available from Amersham, GE Healthcare, Chalfont St. Giles, United Kingdom), diatrizoate meglumine/sodium, iothalamic acid derivatives, iothalamates, ioxithalamic acid derivatives, iothalamate meglumine, metrizoic acid derivatives, iodamide, iodipamide meglumine, ioglycamic acid, dimeric ionic contrast agents, ioxaglic acid derivatives, metrizamide, metrizoate, iopamidol, iohexol, iopromide, iobitridol, iomeprol, iopentol, ioversol, ioxilan, iodixanol, iotrolan, ioxaglate (Hexabrix®, commercially available from Mallinckrodt Imaging, Tyco Healthcare, Mansfield, Mass.), ioxaglate meglumine/sodium, iotrol, iopanoic acid, and organic radiographic iodinated contrast media (ICM) such as modifications of a 2,4,6-tri-iodinated benzene rings including Renografin® (commercially available from Amersham, GE Healthcare, Chalfont St. Giles, United Kingdom), Conray® (commercially available from Mallinckrodt Imaging, Tyco Healthcare, Mansfield, Mass.), iohexol (Omnipaque®, commercially available from GE Healthcare, Chalfont St. Giles, United Kingdom), iopamidol (Isovue®, commercially available from Bracco Diagnostics, Princeton, N.J.), ioversol (Optiray®, commercially available from Mallinckrodt Imaging, Tyco Healthcare, Mansfield, Mass.), and iopromide (Ultravist®, commercially available from Berlex Imaging, Montville, N.J.).

Specific MRI imaging contrast mediums contemplated for use herein include, but are not limited to, gadolinium derivatives and complexes such as gadoteridol, gadoterate meglumine, gadodiamide, and gadopentetate (Magnevist®, commercially available from Berlex Imaging, Montville, N.J.); iron derivatives and complexes; manganese derivatives and complexes such as mangafodipir trisodium; superparamagnetic iron oxide contrast medias; ferumoxides such as FERIDEX® (commercially available from Berlex Imaging, Montville, N.J.); and perfluorocarbons. The MRI imaging contrast mediums may be either positive or negative contrast mediums.

It may be desirable that the MRI imaging contrast mediums comprise complexes of a complexing agent and a metal such as gadolinium, manganese, or iron. Exemplary complexing agents include, but are not limited to, diethylenetriamine-pentaacetic acid (“DTPA”); 1,4,7,10-tetraazacyclododecane-N,N′,N″,N′″-tetraacetic acid (“DOTA”); p-isothiocyanatobenzyl-1,4,7,10-tetraazacyclododecane-1,4,7,10-tetraacetic acid (“p-SCN-Bz-DOTA”); 1,4,7,10-tetraazacyclododecane-N,N′,N″-triacetic acid (“DO3A”); 1,4,7,10-tetraazacyclododecane-1,4,7,10-tetrakis(2-propionic acid) (“DOTMA”); 3,6,9-triaza-12-oxa-3,6,9-tricarboxymethylene-10-carboxy-13-phenyl-tridecanoic acid (“B-19036”); 1,4,7-triazacyclononane-N,N′,N″-triacetic acid (“NOTA”); 1,4,8,11- tetraazacyclotetradecane-N,N′,N″,N′″-tetraacetic acid (“TETA”); triethylene tetraamine hexaacetic acid (“TTHA”); trans-1,2-diaminohexane tetraacetic acid (“CYDTA”); 1,4,7,10-tetraazacyclododecane-1-(2-hydroxypropyl)4,7,10-triacetic acid (“HP-DO3A”); trans-cyclohexane-diamine tetraacetic acid (“CDTA”); trans(1,2)-cyclohexane diethylene triamine pentaacetic acid (“CDTPA”); 1-oxa-4,7,10-triazacyclododecane-N,N′,N″-triacetic acid (“OTTA”); 1,4,7,10-tetraazacyclododecane-1,4,7,10-tetrakis {3-(4-carboxyl)-butanoic acid }; 1,4,7,10-tetraazacyclododecane-1,4,7,10-tetrakis(acetic acid-methyl amide); 1,4,7,10-tetraazacyclododecane-1,4,7,10-tetrakis(methylene phosphonic acid); and derivatives and analogs thereof, particularly protected forms of the compounds.

CT scan imaging contrast mediums contemplated for use in the embodiments include orally, intravenously, and rectally administered mediums. CT scan imaging contrast mediums contemplated for use herein include, but are not limited to, iodine solutions, barium sulfate, mixtures of sodium amidotrizoate and meglumine amidotrizoate (such as Gastrografin®, commercially available from Bristol-Myers Squibb, Princeton, N.J.), and, in general, the imaging contrast mediums mentioned previously in relation to X-rays.

PET scan imaging contrast mediums typically comprise a positron emitting (i.e. radioactive) element incorporated into a carrier such as a complexing agent or a biologically active molecule such as glucose. PET scan imaging contrast mediums contemplated for use in the embodiments include, but are not limited to, complexes and derivatives of positron emitting radioisotopes including, but not limited to, carbon-11, nitrogen-13, oxygen-15, fluorine-18, iron-52, cobalt-55, copper-62, copper-64, bromine-75, bromine-76, technetium-94 m, gallium-68, gallium 66, sellenium-73, bromine-75, bromine-76, iodine-120, iodine-124, and indium-110 m. These radioactive elements may be incorporated into a carrier such as an organic molecule that is fluid at room temperature. Alternatively, these radioisotopes may be complexed with a complexing agent such as the complexing agents previously mentioned in regards to MRI imaging contrast mediums and placed in solution. Because the PET imaging contrast mediums are to be used in the constrained expandable members placed inside the body, it may be preferable to choose PET imaging contrast mediums with short half-lives to reduce the risk to the patient in the event of a rupture of the constrained expandable member. For example, PET imaging contrast mediums with a half-life of about 2 hours such as gallium-68 are preferred.

In another embodiment, the imaging contrast mediums may include a metallic radioisotope including, but not limited to, the isotopes actinium-225, astatine-211, iodine-120, iodine-123, iodine-124, iodine-125, iodine-126, iodine-131, iodine-133, bismuth-212, arsenic-72, bromine-75, bromine-76, bromine-77, indium-110, indium-111, indium-113 m, gallium-67, gallium-68, strontium-83, zirconium-89, ruthenium-95, ruthenium-97, ruthenium-103, ruthenium-105, mercury-107, mercury-203, rhenium-186, rhenium-188, tellurium-121 m, tellurium-122 m, tellurium-125 m, thulium-165, thulium-167, thulium-168, technetium-94 m, technetium-99 m, fluorine-18, silver-111, platinum-197, palladium-109, copper-62, copper-64, copper-67, phosphorus-32, phosphorus-33, yttrium-86, yttrium-90, scandium-47, samarium-153, lutetium-177, rhodium-105, praseodymium-142, praseodymium-143, terbium-161, holmium-166, gold-199, cobalt-57, cobalt-58, chromium-51, iron-59, selenium-75, thallium-201, and ytterbium-169.

In a preferred embodiment, a pressure measurement device may be connected to, and in fluid communication with, the proximate end of the axially concentric bore of the longitudinal element. The pressure measurement device may be used to monitor the pressure of the fluid as it is delivered to the longitudinal element and the connected constrained expandable member. The pressure measurement device may be, for example, a pressure transducer or pressure gauge. Preferably, a pressure set point may be chosen that indicates a safe level of inflation up to which rupture of the constrained expandable member is unlikely to occur.

In another preferred embodiment, a guide shaft having an axially concentric bore, such as a catheter, cannula, or trocar, may be coaxial to the longitudinal element. The guide shaft may facilitate insertion of the longitudinal element and the constrained expandable member. The guide shaft preferably may sheath the longitudinal element and the expandable member. Also, the distal end of the longitudinal element and the expandable member preferably may be extensible beyond the distal end of the guide shaft. In this way, the guide shaft may act as a sheath or sleeve to facilitate insertion of the longitudinal element and the expandable member into the body.

The guide shaft may be inserted into the body before placing the longitudinal element inside of it, or the guide shaft may be inserted into the body with the longitudinal element and constrained expandable member already disposed within it. When the guide shaft reaches or comes near to the intervertebral disc space, the longitudinal element may be extended beyond the distal end of the guide shaft in order to deliver the expandable member to the intervertebral disc space. Thus, the guide shaft may protect the longitudinal element and constrained expandable member during insertion into the body.

The distal end of the longitudinal element and the constrained expandable member may be extended from the distal end of the guide shaft by pushing the longitudinal element into the body while restraining the guide shaft so that the distal end of the longitudinal element and the constrained expandable member are forced out the distal end of the guide shaft. Alternatively, the guide shaft may be retracted away from the body while restraining the longitudinal element, thus forcing the longitudinal element and constrained expandable member out the distal end of the guide shaft. Alternatively, the guide shaft may be a separate element that is first inserted to provide a passageway to the intradiscal space (e.g., a cannula, tissue dilator, etc.) and then the intradisc sizer is advanced through the guide shaft.

As with the longitudinal element, the guide shaft may have an optimally chosen flexibility and diameter or major cross sectional dimension. In a preferred embodiment, because the guide shaft preferably is capable of sheathing the constrained expandable member and the longitudinal element, the diameter or major cross sectional dimension of the axially concentric bore of the guide shaft preferably is large enough to enclose the longitudinal element and the constrained expandable member, in a deflated state. Again like the longitudinal element, the guide shaft may be made of any appropriate material, including medical plastics such as polyvinyl chlorides, polypropylenes, polystyrenes, acetal copolymers, polyphenyl sulfones, polycarbonates, acrylics, silicone polymers, and mixtures and combinations thereof, and medical alloys or metals such as titanium or stainless steel. One of skill in the art will appreciate how to select an appropriate guide shaft in accordance with the guidelines herein.

In another preferred embodiment, the device additionally may comprise a guidewire. The guidewire may be used to guide the longitudinal element during insertion in order to more easily place the longitudinal element at the desired position in the body, for example immediately adjacent to or inside of the intradiscal space. The guidewire may be made from any desirable material, including metals or alloys, and preferably is thin enough to provide the flexibility desired.

Preferably, the longitudinal element may be able to pivot or flex in order to deform the longitudinal element from a linear to a bent or curved configuration. For example, if a guidewire is provided in the longitudinal element and is connected to the distal end of the longitudinal element, proximally retracting the guidewire may cause the longitudinal element to bend or flex. In another embodiment, if a guide shaft is provided, the guide shaft may be bent or flexed in order to cause the longitudinal element disposed therein to bend or flex. A selectively flexible longitudinal element may be advantageous in order to facilitate insertion of the longitudinal element and the constrained expandable member attached thereto into the confines of the intradiscal space. For example, as the distal end of the longitudinal element and the constrained expandable member are inserted into the disc space, it may be desirable to bend or flex the longitudinal member so that it better conforms to the disc space and can reach sufficiently far into the confines of the disc space in order to deliver the expandable member therein.

In another embodiment, there is provided a surgical kit for determining at least one parameter of an intradiscal space. The surgical kit may comprise an intradisc sizer as described herein. For example, the kit may comprise a longitudinal element comprising distal and proximate ends and an axially concentric bore. The kit also may comprise a constrained expandable member having an internal cavity. The constrained expandable member may be either attached to, or capable of being attached to, and in fluid communication with, the distal end of the longitudinal element. The kit further may comprise a dispensing device that is capable of holding a fluid. The dispensing device may be either connected to, or capable of being detachably connected to, and in fluid communication with, the proximate end of the longitudinal element.

Preferably, the kit may further comprise a fluid capable of inflating the constrained expandable member. The fluid may be, for example, a saline solution or an imaging contrast medium. In a more preferred embodiment, the imaging contrast medium may be selected from X-ray, CT scan, MRI, and PET scan imaging contrast mediums.

In a preferred embodiment, the kit also may comprise a guide shaft as described herein. The longitudinal element and constrained expandable member may be capable of being disposed inside of the guide shaft. Furthermore, the kit preferably may include a guidewire. The guidewire may be capable of being positioned within the longitudinal element.

The devices and kits according to the embodiment may be useful for determining the volume, dimensions, and geometry of an intradiscal space. It is preferred that the devices and kits be used in accordance with the methods described hereinafter, although they may be used with other methods of characterizing certain parameters of an intradiscal space that are readily apparent to those skilled in the art

Embodiments also include methods for determining parameters of an intradiscal space utilizing the devices and kits described herein. For example, the intradisc sizer device as described herein may be inserted into the intradiscal space. Following insertion, the constrained expandable member may be inflated with a fluid, such as a saline solution or an imaging contrast medium. The fluid may be delivered to the constrained expandable member by injection from a dispensing device, such as a syringe, into the axially concentric bore of the longitudinal element that is in fluid communication with the constrained expandable member. Injection of the fluid may cause the constrained expandable member to inflate until it reaches substantially the same height as that of the intradiscal space.

One parameter that may be determined by use of the devices, kits, and methods described herein is the volume of the intradiscal space. The volume of the intradiscal space may be estimated by measuring the volume of the inflated constrained expandable member. The volume of the inflated constrained expandable member may be measured by noting the volume displacement of the syringe or other dispensing device by which fluid is delivered to the longitudinal element and constrained expandable member. It may be necessary to subtract from the measurement of the volume displacement of the dispensing device the volume of the concentric bore of the longitudinal element through which the fluid is conducted from the dispensing device to the constrained expandable member. In this way, the volume of the inflated constrained expandable member may be determined.

Estimating the volume of the intradiscal space may aid in selecting a spinal implant to fit the appropriate intradiscal space. Of course, a constrained expandable member with a cross sectional shape similar to the shape of the intradiscal space may yield a volume measurement more closely approximating the volume of the intradiscal space because the constrained expandable member likely will fill more of the intradiscal space when inflated. Therefore, it may be preferable that the constrained expandable member have a “C” or kidney-like planar shape that approximates the shape of the natural intradiscal space. Direct measurement of the intervertebral disc volume using the intradisc sizer and methods disclosed herein may yield a more accurate determination of the volume of the intradiscal space than radiographic measurement alone would yield.

During inflation of the constrained expandable member, it may be desirable to preclude the constrained expandable member from: (i) expanding so much that it distracts the adjacent vertebral bodies too far; and (ii) exerting too much force on adjacent tissue and bone. Furthermore, it may be desirable to ensure that the constrained expandable member has expanded to substantially match the height of the intradiscal space before inflation is stopped. Imaging of the intradiscal space during inflation of the expandable member, or use of a pressure measurement device, may aid a user of the intradisc sizers disclosed herein in accomplishing these goals.

In another preferred embodiment, the constrained expandable member may be inflated with an imaging contrast medium and imaged while inflated to measure or characterize the disc height, foot print, other dimensions, and general geometry or topography of the disc space. One who is skilled in the art will appreciate the existing procedures and methods by which intra-operative radiography may be carried out including 3-dimensional radiographic and ultrasound techniques. The measurements obtained may be used to select a spinal implant prior to implantation. Proper selection of a spinal implant prior to implantation may be advantageous because it can reduce surgical time and increase the likelihood of a desirable clinical result. Measurements of the intradisc space's dimensions and geometry may be made, for example, by manually examining the images created by imaging the inflated constrained expandable member or by computer computation of the dimensions and geometry based on the images obtained.

Parameters that can be measured according to the embodiments include one-dimensional parameters such as the anterior-posterior width, lateral width, and height of the intervertebral disc space. One-dimensional parameters preferably are measured by X-ray (e.g. fluoroscopy). Additionally, two-dimensional parameters such as the cross-sectional areas of the intervertebral disc space perpendicular (i.e. “footprint”) and parallel (i.e. “projected”) to the spinal column can be determined. Simple imaging techniques such as X-ray may be useful to determine the cross-sectional area of the intervertebral disc space parallel to the spinal column, but more advanced imaging techniques such as CT, C-arm fluoroscopy, MRI, and PET technologies preferably are used to determine the cross-sectional area of the disc space perpendicular to the spinal column. Additionally, three-dimensional parameters of the intervertebral disc space such as the volume and geometry (e.g. topography) of the disc space may be determined.

Where a computerized imaging technique is used, parameters of the disc space may be determined by a computer analyzing the obtained images. For example, a computer may directly compute the volume of the intradiscal space or cross-sectional areas of the disc space. In both computational and non-computational imaging techniques, it may be advantageous to include a dimensional reference in the images in order to normalize the observed dimensions of the disc space. For example, a metal structure such as a rod of known dimensions may be placed adjacent to the intradiscal disc space (e.g. on the skin of the patient at a location adjacent to the disc space) prior to imaging such that the rod will appear in the images obtained of the disk space. In this manner, the length of dimensions observed in the images may be normalized to the known length of the dimensional reference.

The inflated constrained expandable member may be imaged with any applicable imaging procedure. Preferred methods of imaging the inflated constrained expandable member include X-ray, CT scan, MRI, and PET scan. In a preferred embodiment, the imaging contrast medium may be selected to correspond to the method of imaging that is to be used. The inflated constrained expandable member may be imaged once or a multiple of times. In another embodiment, more than one imaging procedure may be used. If more than one imaging procedure is to be used, it may be preferable to inflate the constrained expandable member with an imaging contrast medium appropriate for one of the imaging procedures, deflate the constrained expandable member, and then inflate the constrained expandable member again, but with a different imaging contrast medium appropriate for another imaging procedure. This may be repeated for each imaging procedure to be used.

The constrained expandable member may be deflated to facilitate removal of the constrained expandable member from the intradiscal space. The constrained expandable member may be deflated, for example, by reversing the dispensing device such as a syringe that was used to deliver the fluid to the longitudinal element and constrained expandable member, or by drawing a vacuum at the proximal end of the longitudinal element. Following deflation, the constrained expandable member may be removed from the intradiscal space.

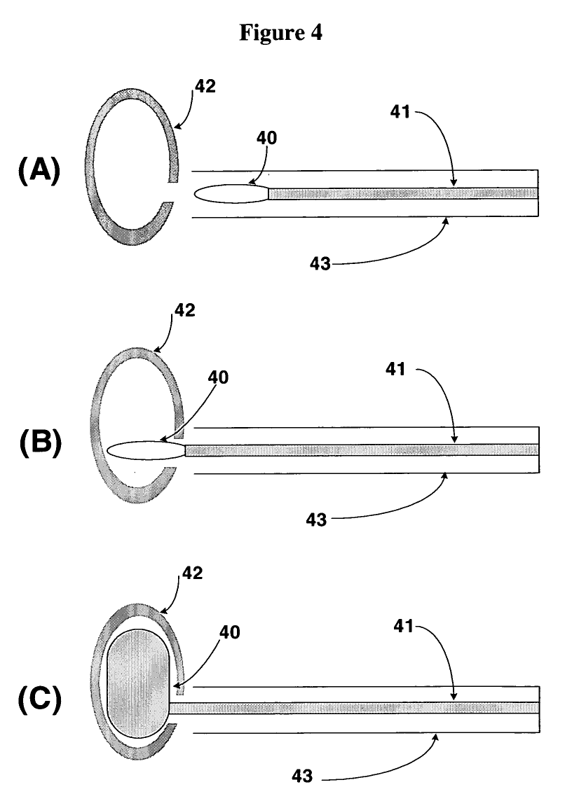

FIG. 4, embodiments A, B, and C, depict an exemplary method of using a device according the embodiments described herein. A constrained expandable member 40 is connected to the distal end of a longitudinal element 41. The constrained expandable member 40 and longitudinal element 41 are sheathed by an optional guide shaft 43. The guide shaft 43 may be approximately coaxial with the longitudinal element 41. Only the distal end of the device is shown, but it is to be understood that the proximate end of the device may include, for example, a syringe or other dispensing device for delivery of a saline solution, imaging contrast medium, or other appropriate fluid. The distal end of the instrument may be advanced to a position that is approximately adjacent to an intradiscal space 42. It is to be understood that the intradiscal space may comprise substantially all of or only a portion of the volume between adjacent vertebrae. The intradiscal space may be created, for example, by partial or full removal of the nucleus of the intervertebral disc.

In embodiment B, the constrained expandable member 40 and distal end of the longitudinal element 41 are extended beyond the distal end of the guide shaft 43 to move towards and, at least in the case of the constrained expandable member, into the intradiscal space. A saline solution, air, an imaging contrast medium, or other appropriate fluid may be delivered to the constrained expandable member to cause it to inflate. Embodiment C depicts the constrained expandable member 40 inflated until it has substantially occupied the intradiscal space 42.

In another embodiment, there is provided a method of implanting a spinal implant. According to the method, at least a portion of a nucleus of an intervertebral disc may be removed to evacuate at least a portion of the intradiscal space. For example, a diseased or damaged portion of the nucleus or annulus of the intervertebral disc may be removed before insertion of the constrained expandable member. Alternatively, a complete nucleotomy or discectomy may be performed to remove the nucleus or entire intervertebral disc before insertion of the constrained expandable member. One who is skilled in the art will appreciate how a portion or all of the nucleus is to be removed prior to insertion of the constrained expandable member.

A constrained expandable member as described herein may be inserted into the intervertebral disc space and inflated with a fluid. The volume of the fluid used to inflate the constrained expandable member may be measured. The inflated constrained expandable member then may be deflated and removed from the intradiscal space. A spinal implant may be selected based at least on the volume of fluid used to inflate the constrained expandable member. The selected spinal implant then may be implanted into the disc space. This method reduces operation time by eliminating or significantly reducing the trial-and-error typically needed to select the appropriately-sized spinal implant.

The fluid used to inflate the constrained expandable member may selected from saline solution and an imaging contrast medium. If an imaging contrast medium (e.g., X-ray, CT scan, MRI, and PET scan mediums) is used, then the constrained expandable member preferably may be imaged while inflated. For example, an X-ray, CT scan, MRI, and PET scan may be performed on the inflated constrained expandable member. Imaging of the intradiscal space and constrained expandable member may allow additional parameters, such as the height of the intradiscal space, to be determined.

The invention has been described with reference to particularly preferred embodiments and examples. Those skilled in the art will appreciate that various modifications may be made to the invention without departing from the spirit and scope thereof.

What is claimed is:

1. An intradisc sizer device for determining at least one parameter of an intradiscal space, comprising:
a longitudinal element having distal and proximate ends and an axially concentric bore;
a constrained expandable member comprising an internal cavity, said member connected to and in fluid communication with the distal end of the longitudinal element; and,
a dispensing device capable of holding a fluid and adapted to be connected to and in fluid communication with the proximate end of the longitudinal element.

2. The device of claim 1, wherein the constrained expandable member comprises a polymeric material selected from the group consisting of polyethylene terephthalates, polyolefins, polyurethanes, nylon, polyvinyl chloride, silicone, polyetherketone, polylactide, polyglycolide, poly(lactide-co-glycolide), poly(dioxanone), poly([epsilon]-caprolactone), poly(hydroxylbutyrate), poly(hydroxylvalerate), tyrosine-based polycarbonate, polypropylene fumarate, and mixtures and combinations thereof.

3. The device of claim 1, wherein the constrained expandable member has a planar view, when the expandable member is substantially inflated, having a shape selected from the group consisting of a circle, ellipse, rectangle with rounded corners, kidney, and “C”-shaped.

4. The device of claim 1, wherein the dispensing device is a syringe graduated by volume.

5. The device of claim 1, wherein the fluid is selected from saline solution, air, and an imaging contrast medium.

6. The device of claim 5, wherein the imaging contrast medium is selected from the group consisting of X-ray, C-arm fluoroscopy, CT scan, MRI, and PET scan imaging contrast mediums.

7. The device of claim 1, wherein the longitudinal element is capable of moving between a linear and a curved configuration.

8. The device of claim 1, further comprising a guidewire positioned within the longitudinal element.

9. The device of claim 1, further comprising a guide shaft having an axially concentric bore, wherein the longitudinal element and constrained expandable member are capable of being positioned within the guide shaft.

10. The device of claim 9, wherein the constrained expandable member and the distal end of the longitudinal element are capable of extending beyond a distal end of the guide shaft.

11. A kit for determining at least one parameter of an intradiscal space, comprising:
a longitudinal element having distal and proximate ends and an axially concentric bore;
a constrained expandable member comprising an internal cavity, said member capable of being connected to and in fluid communication with the distal end of the longitudinal element; and,
a dispensing device capable of holding a fluid, said device capable of being connected to and in fluid communication with the proximate end of the longitudinal element.

12. The kit of claim 11, further comprising a fluid capable of inflating the constrained expandable member.

13. The kit of claim 12, wherein the fluid is selected from a saline solution and an imaging contrast medium.

14. The kit of claim 13, wherein the imaging contrast medium is selected from the group consisting of X-ray, CT scan, MRI, and PET scan imaging contrast mediums.

15. The kit of claim 11, further comprising a guidewire that is capable of being positioned within the longitudinal element.

16. The kit of claim 11, further comprising a guide shaft having an axially concentric bore, wherein the longitudinal element and constrained expandable member are capable of being positioned within the guide shaft.

17. A method for determining at least one parameter of an intradiscal space, comprising:
inserting a constrained expandable member into the intradiscal space;
inflating the constrained expandable member with a fluid;
measuring the volume of fluid used to inflate the constrained expandable member;
deflating the constrained expandable member; and,
removing the constrained expandable member from the intradiscal space.

18. The method of claim 17, wherein the fluid is selected from saline solution, air, and an imaging contrast medium.

19. The method of claim 18, wherein the fluid is an imaging contrast medium.

20. The method of claim 19, wherein the imaging contrast medium is selected from the group consisting of X-ray, CT scan, MRI, and PET scan imaging contrast mediums.

21. The method of claim 19, further comprising imaging the intradiscal space while the constrained expandable member is inflated with the imaging contrast medium.

22. The method of claim 21, wherein imaging the intradiscal space comprises an imaging procedure selected from the group consisting of X-ray, CT scan, MRI, and PET scans.

23. A method of implanting a spinal implant in an intradiscal space, comprising:
removing at least a portion of a nucleus of an intervertebral disc;
inserting a constrained expandable member into the intradiscal space;
inflating the constrained expandable member with a fluid;
measuring the volume of the fluid used to inflate the constrained expandable member;
deflating the constrained expandable member;
removing the constrained expandable member from the intradiscal space;
selecting a spinal implant based at least on the volume of fluid used to inflate the constrained expandable member; and,
implanting the spinal implant.

24. The method of claim 23, wherein the fluid is selected from saline solution, air, and an imaging contrast medium.

25. The method of claim 24, wherein the fluid is an imaging contrast medium.

26. The method of claim 25, wherein the imaging contrast medium is selected from the group consisting of X-ray, CT scan, MRI, and PET scan imaging contrast mediums.

27. The method of claim 25, further comprising imaging the intradiscal space while the constrained expandable member is inflated with the imaging contrast medium.

28. The method of claim 27, wherein imaging the intradiscal space comprises an imaging procedure selected from the group consisting of X-ray, CT scan, MRI, and PET scans.

29. The method of claim 23, wherein the constrained expandable member has a shape similar to the shape of the spinal implant.
